(19) United States
(12) Patent Application Publication
(10) Pub. No.: US 2014/0335586 A1
(43) Pub. Date: Nov. 13, 2014

(54) SYSTEM AND METHOD FOR CONVERTING ACIDS TO HYDROCARBON FUELS

(71) Applicant: EE-TERRABON BIOFUELS LLC, New Braunfels, TX (US)

(72) Inventors: Jubo ZHANG, College Station, TX (US); Cesar B. GRANDA, College Station, TX (US)

(73) Assignee: EE-TERRABON BIOFUELS LLC, New Braunfels, TX (US)

(21) Appl. No.: 14/267,841

(22) Filed: May 1, 2014

Related U.S. Application Data
(60) Provisional application No. 61/822,195, filed on May 10, 2013.

(51) Int. Cl. C07C 1/22; C12P 5/02; C10G 1/00

(52) U.S. Cl. CPC C07C 1/22; C10G 1/002; C12P 5/02
USPC 435/167; 585/240

(57) ABSTRACT

Herein disclosed is a method comprising: converting at least a portion of the biomass into medium-chain fatty acids or carboxylic acids ranging from C4 to C9; reacting at least a portion of the medium-chain fatty acids or carboxylic acids in a ketonization reactor to produce a ketonization product; and reacting at least a portion of the ketonization product in a hydrodeoxygenation reactor to remove substantially all oxygen and produce a hydrodeoxygenation product comprising n-paraffins. Herein also disclosed is a system comprising: a fermentation unit to convert the biomass into medium-chain fatty acids or carboxylic acids ranging from C4 to C9; a ketonization reactor configured to receive at least a portion of the medium-chain fatty acids or carboxylic acids and to produce a ketonization product; and a hydrodeoxygenation reactor configured to receive at least a portion of the ketonization product and to produce a hydrodeoxygenation product comprising n-paraffins.

### CROSS-REFERENCE TO RELATED APPLICATIONS

This application claims the benefit under 35 U.S.C. §119(e) of U.S. Provisional Patent Application No. 61/822,195, filed May 10, 2013, the disclosure of which is hereby incorporated herein by reference.

### STATEMENT REGARDING FEDERALLY SPONSORED RESEARCH OR DEVELOPMENT

Not Applicable.

### BACKGROUND

1. Technical Field

This disclosure generally relates to system and method for producing fuels. More particularly, this disclosure relates to system and method for converting medium length acids to hydrocarbon fuels.

2. Background of the Invention

Mixed culture fermentation from renewable biomass resources is one of the most economically competitive methods to convert biomass materials to renewable chemicals and fuels. These microorganisms generally produce a mixture of carboxylic acids, which are short- and medium-chain fatty acids (MCFAs) ranging from acetic acid (C2) all the way to octanoic (C8) and even nonanoic acid (C9). Through controlled fermentation and selective recovery and with or without the addition of certain reducing compounds like ethanol or hydrogen, the product profile of the carboxylic acid products can be adjusted to be in the range of C4 to C9 carbon numbers. This mixture of C4-C9 MCFAs may be recovered from fermentation broth using several methods, such as acidification and extraction. MCFAs are high-value products in the chemical market for applications such as in detergents, cosmetics, food additives, paints, etc. However, MCFA can also be transformed to hydrocarbon fuel products through catalytic chemical processing.

To convert fatty acids to hydrocarbons, regardless of the method, the oxygen content (36-20 wt % oxygen for C4-C9 MCFA) needs to be reduced significantly and ideally close to zero. The straightforward method is hydrotreating/hydrogenating the carboxylic acid or its esters with noble-metal catalysts. However, large amounts of H₂ are required to combine with oxygen to form water byproduct for the removal of the oxygen, a process known as hydro-deoxygenation (HDO). In addition, the hydrotreating/hydrogenating process of carboxylic acid or esters does not only involve HDO but also de-carboxylation, where the carboxylic acid functional group is removed in every molecule of acid as carbon dioxide, thus producing shorter n-paraffins (e.g., C3-C8). The carbon dioxide produced may then be further reverse shifted to carbon monoxide and carbon monoxide may be methanated to methane, consuming even larger quantities of H₂. The presence of carbon monoxide is also known to cause many problems in the reactor such as catalyst inhibition, deactivation and increased temperature.

Conversion of mixed short- and medium-chain fatty acids have been proposed in the past but such conversion requires extensive oligomerization (i.e., elongation of the carbon chain) to achieve the proper final hydrocarbon length. MCFAs, on the other hand, either already have the proper length or they only require the joining or dimerization of two molecules to achieve the proper length. Thus, there is a need in the art for new, improved systems and processes for the production of hydrocarbon fuels from medium-chain fatty acids, wherein the medium-chain fatty acids may be produced from renewable resources, such as, biomass.

### SUMMARY

Herein disclosed is a method for producing hydrocarbon fuels from biomass, the method comprising: converting at least a portion of the biomass into medium-chain fatty acids or carboxylic acids ranging from C4 to C9; reacting at least a portion of the medium-chain fatty acids or carboxylic acids in a ketonization reactor to produce a ketonization product; and reacting at least a portion of the ketonization product in a hydrodeoxygenation reactor to remove substantially all oxygen and produce a hydrodeoxygenation product comprising n-paraffins.

In some embodiments, the medium-chain fatty acids or carboxylic acids ranging from C4 to C9 are produced via controlled fermentation. In some embodiments, the controlled fermentation comprises introducing a compound into the fermentation to effect chain elongation by microorganisms, wherein the compound is selected from the group consisting of ethanol, lactic acid, hydrogen and combinations thereof. In some embodiments, the controlled fermentation comprises separating C2-C3 fatty acids or carboxylic acids from the fermentation product and recycling them to the fermentation for chain elongation. In some embodiments, conversion of biomass to acids and chain elongation take place in separate reactors or separate series of reactors operated under different conditions.

In some embodiments, ketonization takes place at a temperature in the range of from 200° C. to 600° C. In some embodiments, ketonization takes place at a temperature in the range of from 300° C. to 500° C. In some embodiments, ketonization takes place at a pressure in the range of from 0.1 atm to 10 atm. In some embodiments, ketonization utilizes a catalyst comprising oxides of cerium, oxides of manganese, oxides of calcium, oxides of magnesium, or combinations thereof. In some embodiments, the ketonization step produces a ketone yield of greater than 90%.

In some embodiments, hydrodeoxygenation takes place at a temperature in the range of from 0° C. to 600° C. In some embodiments, hydrodeoxygenation takes place at a temperature in the range from 200° C. to 500° C. In some embodiments, hydrodeoxygenation takes place at a pressure in the range of from 0.1 to 250 atm. In some embodiments, hydrodeoxygenation utilizes a catalyst comprising cobalt, molybdenum, nickel, palladium, ruthenium, or combinations thereof. In some embodiments, sulfur is added to stabilize the hydrodeoxygenation catalyst.

In some embodiments, the ketonization product comprises C7-C17 ketones. In some embodiments, the hydrodeoxygenation product comprises C7-C17 n-paraffins.

In some embodiments, the hydrodeoxygenation product is further processed to produce transportation fuels or drop-in fuels. In some embodiments, the hydrodeoxygenation product is further processed to produce diesel or jet fuel or both. In some embodiments, the hydrodeoxygenation product is further processed via hydroisomerization, hydrocracking, catalytic reforming, fractionation, distillation, or combinations thereof.

Herein also disclosed is a system for producing hydrocarbon fuels from biomass, the system comprising: a fermentation unit to convert the biomass into medium-chain fatty acids or carboxylic acids ranging from C4 to C9; a ketonization reactor configured to receive at least a portion of the medium-chain fatty acids or carboxylic acids and to produce a ketonization product; and a hydrodeoxygenation reactor configured to receive at least a portion of the ketonization product and to produce a hydrodeoxygenation product comprising n-paraffins. In some embodiments, the system further comprises a hydroisomerization unit configured to receive at least a portion of the hydrodeoxygenation product. In some embodiments, the system further comprises a distillation unit downstream of the hydroisomerization unit to produce jet fuel or aviation gasoline or both. In some embodiments, the system further comprises a distillation unit configured to receive at least a portion of the hydrodeoxygenation product to produce diesel. In some embodiments, the system further comprises a catalytic reforming unit downstream of the distillation unit to produce gasoline.

Further disclosed herein is a method for producing hydrocarbon fuels from biomass, the method comprising: converting at least a portion of the biomass into fatty acids or carboxylic acids by means of fermentation; and reacting at least a portion of the fatty acids or carboxylic acids to remove substantially all oxygen and produce n-paraffins by means of catalytic hydrodeoxygenation or decarboxylation or both. In some embodiments, hydrodeoxygenation or decarboxylation takes place at a temperature in the range of from 0° C. to 600° C. In some embodiments, hydrodeoxygenation or decarboxylation takes place at a temperature in the range of from 200° C. to 500° C. In some embodiments, hydrodeoxygenation or decarboxylation takes place at a pressure in the range of from 0.1 to 250 atm. In some embodiments, hydrodeoxygenation or decarboxylation utilizes a catalyst comprising cobalt, molybdenum, nickel, palladium, ruthenium, or combinations thereof.

In some embodiments, hydrodeoxygenation or decarboxylation product is further processed to produce transportation fuels or drop-in fuels. In some embodiments, hydrodeoxygenation or decarboxylation product is further processed to produce diesel or jet fuel or both. In some embodiments, the hydrodeoxygenation or decarboxylation product is further processed via hydroisomerization, hydrocracking, catalytic reforming, fractionation, distillation, or combinations thereof.

Herein disclosed is a method for producing hydrocarbon fuels from biomass, the method comprising: converting at least a portion of the biomass into medium-chain fatty acids or carboxylic acids ranging from C4 to C9; reacting at least a portion of the medium-chain fatty acids or carboxylic acids in a hydrogenation reactor to produce alcohols; and reacting at least a portion of the alcohols in a dimerization or trimerization reactor to remove substantially all oxygen and produce olefins and to dimerize or trimerize such olefins to produce a dimerization/trimerization product comprising n-paraffins.

Further disclosed herein is a method for producing hydrocarbon fuels from biomass, the method comprising: converting at least a portion of the biomass into medium-chain fatty acids or carboxylic acids ranging from C4 to C9; reacting at least a portion of the medium-chain fatty acids or carboxylic acids in a hydrogenation reactor to produce alcohols; and reacting at least a portion of the alcohols in a dimerization or trimerization reactor to remove substantially all oxygen and produce olefins and to dimerize and trimerize such olefins to produce a dimerization/trimerization product comprising n-paraffins.

In some embodiments, the hydrogenation of the medium-chain fatty acids or carboxylic acids occurs by reacting the acids with an alcohol or an olefin to form a esters, and then hydrogenolyzing the ester to form alcohols. In some embodiments, the dimerization/trimerization product comprises C8-C18 paraffins. In some embodiments, the dimerization/trimerization product is further processed to produce transportation fuels or drop-in fuels. In some embodiments, the dimerization/trimerization product is further processed to produce diesel or jet fuel or both. In some embodiments, the method further comprises processing the dimerization/trimerization product via hydroisomerization, hydrocracking, catalytic reforming, fractionation, distillation, or combinations thereof.

Although specific advantages are enumerated herein, various embodiments may include all, some, or none of the enumerated advantages. Additionally, other technical advantages may become readily apparent to one of ordinary skill in the art after review of the following figures and description.

### BRIEF DESCRIPTION OF THE DRAWINGS

For a more complete understanding of example embodiments of the present invention and its advantages, reference is now made to the following description, taken in conjunction with the accompanying drawings, in which:

FIG. 1 is a block diagram showing downstream chemical process to upgrade the MCFA to transportation fuel products, according to an embodiment;

FIG. 2 illustrates a mechanism in the ketonization reaction where σ are the acidic sites on the catalyst;

FIG. 3 illustrate reaction pathways for carboxylic acids at high temperatures, according to an embodiment;

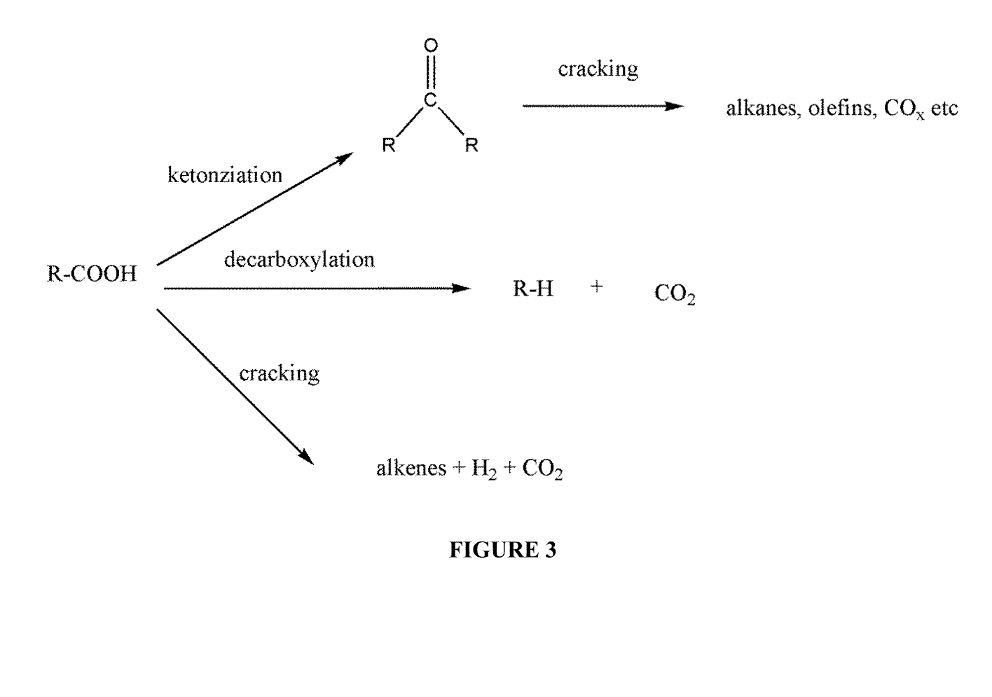

FIG. 4 is from Glinski et al, 2000 and shows the yield of acetone in a long time experiment over 20 wt-% CeO2/SiO2, T=623K, LHSV=3 cm³ g⁻¹ h⁻¹;

FIG. 5 is a process flow diagram, illustrating single pass of MCFA ketonization over metal oxide catalysts, according to an embodiment;

FIG. 6 is a process flow diagram, illustrating ketonization of MCFA with some product recycle, according to an embodiment;

FIG. 7 is illustrate chemical pathways for converting ketones to hydrocarbons, according to an embodiment;

FIG. 8 is a process flow diagram, illustrating a process for the conversion of ketones to hydrocarbons, according to an embodiment;

FIG. 9 shows an example of catalytic reforming reactions, according to an embodiment;

FIG. 10 is from Speight 2007 and shows an example of catalytic reforming process;

FIG. 11 is a block diagram showing an integrated process for refining a product from the hydrodeoxygenation of ketones to transportation fuels, according to an embodiment.

FIG. 12 is a block diagram showing an integrated process for refining a product from the dimerization or trimerization of olefins obtained from primary alcohols to transportation fuels, according to an embodiment.

### DETAILED DESCRIPTION

Overview.

In this disclosure, unless otherwise specified, short-chain organic molecules are considered to contain 1-3 carbon atoms; medium-chain organic molecules are considered to contain 4-9 carbon atoms; and long-chain organic molecules are considered to contain no less than 10 carbon atoms. The word biomass means any material of biological or organic origin that may be biologically converted or degraded by bio-catalysts such as enzymes and/or microorganisms.

Herein disclosed is a method to upgrade medium length acids (e.g., MCFAs or carboxylic medium length acids) to advanced hydrocarbons used for transportation fuels such as jet fuel, diesel, gasoline and aviation gasoline. In this disclosure, MCFA is often used for ease of reference to represent medium length acids. As one skilled in the art would appreciate, the method/process disclosed herein is not limited only to MCFAs.

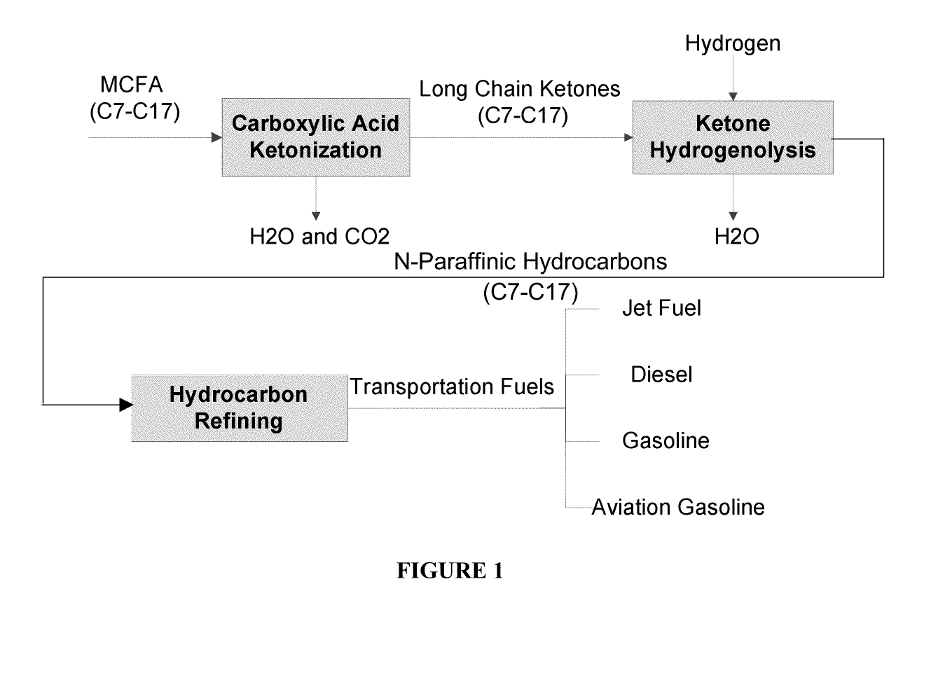

In an embodiment, FIG. 1 is a block diagram showing three major steps for MCFA upgrading. The ketonization step converts the carboxylic acid to long chain ketone products as well as CO₂ and H₂O byproducts. This step reduces the oxygen content without consumption of hydrogen, and elongates the chemical chains through dimerization for use as precursors preferably to fuels other than gasoline, e.g., diesel and jet fuel. The long chain mixed ketones (C7-C17) are also valuable chemical products with application in food additives, cosmetics, solvents. But they may also be further upgraded to hydrocarbon drop-in fuels. After the oxygen content is reduced significantly in the long chain ketones (0.05-2 wt % Oxygen in C7-C17 ketones) from the oxygen content in fatty acids (20-40%) through ketonization, then the hydrodeoxygenation (HDO) process (i.e., hydrogenolysis of ketones to hydrocarbons) is applied using standard refinery CoMo or NiMo catalysts as well as other newly developed catalysts, which produce normal linear paraffins. The resulting normal paraffinic hydrocarbons are in the range of 7 to 17 carbon numbers, which may be further upgraded into gasoline, jet fuel and diesel fractions using known technologies in the petroleum industry.

In some embodiments, medium chain fatty acids in the range of C4-C9 produced through controlled mixed-acid fermentation in the upstream are upgraded to transportation hydrocarbon fuels effectively and efficiently through three major steps:
(1) Ketonic decarboxylation of MCFAs over metal oxides catalysts, such as but not limited to, manganese and cerium oxides, producing a mixture of longer-chain ketones in the range of C7-C17. The catalysts and process conditions (e.g., partial pressure, temperature, and space velocities) are selected to maximize the ketone yield (e.g., >90%). Cerium and manganese oxides are used, in some cases, for ketonization running at atmospheric pressure, at temperatures ranging from 300° C. to 500° C. and liquid-hourly space velocities in the 1 to 5 cm³/(g cat.-h) range.
(2) Hydrogenolysis or hydrodeoxygenation (HDO) of the C7-C17 mixed ketones over, such as but not limited to, CoMo/Al₂O₃ or NiMo/Al₂O₃ in the presence of high pressure hydrogen, producing hydrocarbon HDO product of C7-C17 n-paraffins. For these catalysts, the sulfided state of these catalysts is preferred. In some cases, sulfur is added to stabilize the HDO catalyst if the feed being processed does not contain sulfur.
(3) Refining of paraffinic HDO product to transportation fuels. Various processes and systems may be configured according to the desired products as known to one skilled in the art. The on-specification kerosene-type jet fuel or diesel fuel, gasoline or aviation gasoline may be produced from the HDO product specifically by selecting the appropriate processes, such as but not limited to, hydroisomerization, hydrocracking, catalytic reforming, and fractionation/distillation.

Ketonization.

Calcium or other metal carboxylate salts may be pyrolyzed at elevated temperature to make ketones. For example, acetone may be manufactured through dry distillation of calcium acetate with high yield at a temperature of about 300-400° C. The reaction occurs as follows:

R1-COO—Ca—OOC—R2→R1-CO—R2+CaCO₃ Eq. (1)

This process that converts carboxylic acid to metal salt followed by its pyrolysis is unattractive as the handling of solids makes it unfavorable for scaling up and operational robustness. Thus, direct conversion of carboxylic acids to ketones through catalytic reaction is more advantageous for upgrading the medium-chain fatty acids, obtained, for example, from a fermentation process. Different from calcium carboxylates pyrolysis with solid calcium carbonate as byproduct, ketonization of carboxylic acids produces ketones with water and carbon dioxide. The reaction is as follows:

R1-COOH+R2-COOH→R1-CO—R2+CO₂+H₂O Eq. (2)

The liquid-vapor phase reaction is convenient for a continuous process and scale-up without solids handling. However, if the reaction is not properly controlled, the reaction of free acids at elevated temperatures normally results in complicated reactions and yields little ketone product. For example, the direct decarboxylation of the free acids to olefins and carbon dioxide is one possible side reaction:

R—CH₂—CH₂—COOH→R—CH═CH₂+CO₂+H₂ Eq. (3)

Thus, in various embodiments, the reaction is controlled with the proper catalysts and at the proper temperatures, partial pressures of the reacting acids and space velocities to maximize ketone yield with high acid conversion for the ketonization process. In some cases, high ketone yields are obtained with cerium and manganese oxide catalysts in silica, alumina and titania supports, at a temperature in range of from 300° C. to 500° C., liquid-hourly space velocities in the 1 to 5 cm³/(g cat.-h) range and partial pressure of less than 1 atm.

Ketonization Process.

In an embodiment, the ketonization process utilizes catalysts, including oxides of various metals, e.g., magnesium, calcium, aluminum, copper, zinc, zirconium, lead, iron, cobalt, manganese, cadmium, niobium, thorium, cerium and other lanthanides. In some cases, ketonization takes place in the presence of an oxide catalyst supported on SiO₂, Al₂O₃ and/or TiO₂. In some cases, MnO₂ or CeO₂ are used to convert acids to ketones with a high yield of, for example, greater than 95%. In some cases, the ketonization catalyst is 20% MnO₂ or CeO₂ on alumina support.

The ketonization process reduces the oxygen content significantly without consumption of hydrogen. For example, to convert two hexanoic acid (C6) to one undecanone (C11), there is a small carbon loss (one of 12 carbons or 8%, i.e., 1 mole of CO₂ per every two moles of acid) with the removal of 75% of the oxygen.

Ketonization is an economically attractive way to upgrade the medium chain fatty acid. As shown in the equation below, one long chain ketone may be made from two molecules of carboxylic acids with side products of carbon dioxide and water.

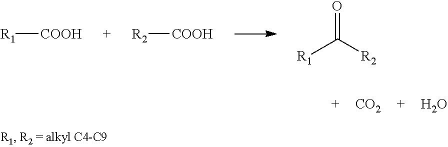

When R1 and R2 are the same alkyl group (one single pure acid is the reactant), only one symmetric ketone is produced. For example, hexanoic acid is reacted to form only one type of ketone as 6-undecanone. However, when mixed acids of C4-C9 carbon chain length are used as reactants, a mixture of ketones in the C7 to C17 range (Table 1) are expected by combining the R1 and R2 groups. When the C4-C9 mixed acids of certain distribution profile is used, the product distribution profile of mixed ketones C7-C17 may be predicted based on statistical theory of binomial distribution, which assumes the same reactivity of similar carboxylic acids where homo- or cross ketonization of any two molecules of acid has the same probability.

TABLE 1
Medium chain fatty acid and ketonization products.

| Medium Chain Fatty Acids | | Long chain Ketones | |
|---|---|---|---|
| C # | Acids | C # | Ketones |
| 4 | CH₃CH₂CH₂COOH | 7 | 4-heptanone |
| 5 | CH₃(CH₂)₃COOH | 8 | 4-octanone |
| 6 | CH₃(CH₂)₄COOH | 9 | 5-nonanone |
| 7 | CH₃(CH₂)₅COOH | 10 | 4-decanone, 5-decanone |
| 8 | CH₃(CH₂)₆COOH | 11 | 4-undecanone, 5-undecanone, |
| | | | 6-undecanone |
| 9 | CH₃(CH₂)₇COOH | 12 | 4-dodecanone, 5-dodecanone, |
| | | | 6-dodecanone |
| | | 13 | 5-tridecanone, 6-tridecanone, |
| | | | 7-tridecanone |
| | | 14 | 6-tetradecanone, 7-tetradecanone |
| | | 15 | 8-pentadecanone |
| | | 16 | 8-hexadecanone |
| | | 17 | 9-heptadecanone |

The reactivity of long chain acids decreases with increasing chain length. The heavy acids generally require higher temperatures to achieve the same conversion as the lower acids. However, higher temperatures increase the propensity for cracking as well as other side reactions. The ketone yield or selectivity of MCFAs with different chain length may vary depending on the catalyst, temperature, residence time, and alkyl chain structures. Considering the nature of the mixture of MCFA feed, a product of mixed long-chain ketones in the range of C7 to C17 is expected with product distribution profile skewed towards lower molecular weight from the theoretical prediction. Along with ketones as the major product, some acid and ketone cracking products, such as olefins and hydrocarbons, are also expected. Discussion of catalysts and process conditions is presented herein below to achieve high ketone yield with high acid conversion and selectivity.

Process Conditions.

The process performance is mostly concerned with acid conversion and ketone yield to catalytically convert MCFAs into longer-chain ketones in a continuous process. However, other process economics factors such as catalyst cost and longevity, product stream separation and recycle, need to be considered to scale up the production process in a commercial plant.

Thermodynamics.

Some thermodynamic calculation results are presented in Tables 2 and 3 from original enthalpy and entropy data from Yaws' Chemical Properties Handbook. The ketonization of acetic acid to acetone with water and carbon dioxide is an endothermic reaction around the typical catalytic reaction temperature (˜700 K). The large entropy change of the reaction drives the reaction to be highly favorable as demonstrated by the negative Gibbs energy change and thus large reaction equilibrium constants.

Shift of the feed from acetic acid to heavy acid such as hexanoic acid results in slight negative enthalpy change of reaction (ΔHr<0, i.e., exothermic reaction), and more favorable reaction with larger equilibrium constants. It would be, therefore, concluded from the thermodynamics that mixed C4-C9 acids have a more complete reaction than acetic acid, which is, in some cases >95% conversion and yield and with such conversion being accomplished over many catalyst systems at a temperature of 300-400° C.

More thermodynamic calculations show that high conversion of hexanoic acid (>95%) in a wide range of temperatures and pressures is maintained when the reaction reaches equilibrium. Co-feeding of water and other diluents also has little effect on the acid conversion due to the large reaction equilibrium constants. As such, it is contemplated that ketonization of MCFAs has no thermodynamic constraints to prevent high conversion of the acid.

TABLE 2
Thermodynamic data on acetic acid ketonization.

| Reac- | 2 CH₃COOH = | | | |
|---|---|---|---|---|
| tion | CH₃COCH₃ + CO₂ + H₂O | | | |
| Tem- | Enthalpy | Entropy | Gibbs energy | reaction |
| pera- | change of | change of | change of | equilibrium |
| ture | reaction, ΔH_{R} | reaction, ΔS_{R} | reaction, ΔG_{R} | constant, Kₚ |
| (K) | (kJ/mol) | (J/mol K) | (kJ/mol) | (atm) |
| 300 | 12.5 | 128.4 | −26.02 | 3.35E+04 |
| 500 | 14.1 | 138.1 | −54.95 | 5.42E+05 |
| 700 | 13.3 | 138.2 | −83.44 | 1.66E+06 |

TABLE 3
Thermodynamic data on hexanoic acid ketonization.

| Reac- | 2 C₅H₁₁COOH = | | | |
|---|---|---|---|---|
| tion | C₅H₁₁COC₅H₁₁ + CO₂ + H₂O | | | |
| Tem- | Enthalpy | Entropy | Gibbs energy | reaction |
| pera- | change of | change of | change of | equilibrium |
| ture | reaction, ΔH_{R} | reaction, ΔS_{R} | reaction, ΔG_{R} | constant, Kₚ |
| (K) | (kJ/mol) | (J/mol K) | (kJ/mol) | (atm) |
| 300 | −0.6 | 159.1 | −48.33 | 2.54E+08 |
| 500 | −1 | 163 | −82.5 | 4.06E+08 |
| 700 | −2.1 | 163.4 | −116.48 | 4.81E+08 |

Kinetics.

Acid conversion and the ketone yield are dependent on the catalyst systems and acid structure as well as other materials being co-fed. Low-molecular weight acids, e.g., acetic acid, have high conversion and selectivity to ketone at lower temperature and high space velocities compared to heavy fatty acids. The branched acids, especially with alpha-alkyl groups, tend to have low conversion over metal oxide catalysts.

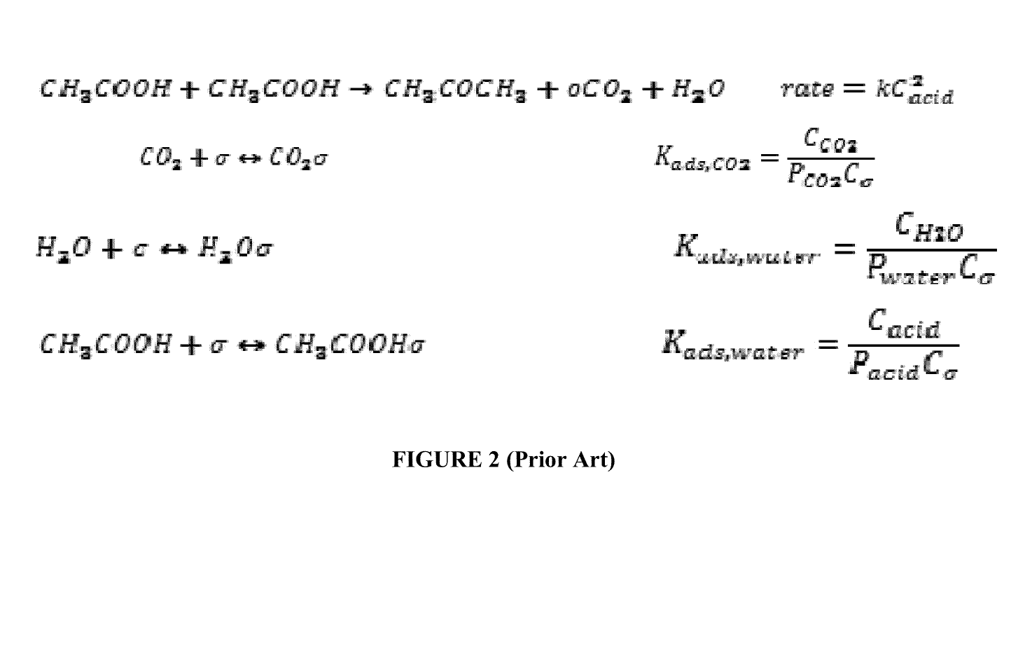

A reaction rate model was proposed based on competitive absorption of acid, CO₂ and water on the active metal oxide center (FIG. 2) based on Langmuir-Hinshelwood-Hougen-Watson theory. Kinetic rates were found to be dependent on acid coverage on the surface of the catalyst. The absorption of ketone products on the active center is negligible. However, absorption of water and CO₂ is strongly competitive against the acid absorption.

Thus, lower reactivity of heavy acid may be explained from kinetic theory. The diffusion and absorption of long-chain acids is relatively slow compared to the small acetic acid. The details of the kinetic model for each carboxylic acid on a specific catalyst such as adsorption constants and second order reaction rate constants are ideal data for a process design on the MCFAs ketonization. For hexanoic acid ketonization over ceria-zirconia catalyst, some observations are as follows: First, the ketonization rate has strong temperature dependence with activation energy of 134 kJ/mol. (In comparison, acid-alcohol esterification has activation energy of 28 kJ/mol on the same catalyst surface) Thus, high temperature is favored for ketonization. Second, the ketonization rate shifts from second-order to zero order as the partial pressure of acids increases. After the catalyst surface is saturated by acids, increase of acid pressure does not help the reaction rate. Third, strong inhibiting effect of water and carbon dioxide is demonstrated while co-feeding ketone does not affect the reaction rate significantly. So minimizing partial pressure of water and CO₂ is beneficial for process performance.

Other than the kinetic factors that affect the acid conversion rate, other competitive side reactions also contribute to the low ketone yield. At temperatures as high as 400-500° C., decarboxylation of acids to form alkanes and CO₂ could be catalyzed on some basic metal oxide surface. Acid cracking through radical mechanism can become significant at high temperature. Cracking of heavy ketones at high temperature can lower the final ketone yield. Thus, the catalyst needs to have high ketonization selectivity on metal oxide surface compared to other cracking reaction pathways.

Catalysts. In some embodiments, CeO2 or MnO₂ on silica is used for ketonization of carboxylic acids, especially for acetic acid. In some embodiments, ketonization of hexanoic acid takes place on a catalyst of formula Ce_{0.5}Zr_{0.5}O₂. In some embodiments, the CeO₂ based CeO₂—Mn₂O₃ is used for ketonization of propanoic acid and cross ketonization of mixed acids. In an embodiment, a catalyst containing alkaline earth metal oxides (either CaO or MgO) has better acid conversion and ketone yield compared to other metal oxides like CeO₂ and ZrO₂, when the feeding acid of n-caprylic acid is diluted in a stream with 95% N₂ gas. Transition metal oxides like CeO₂ tend to have more stability; however, alkaline earth metal oxides are among the cheapest materials.

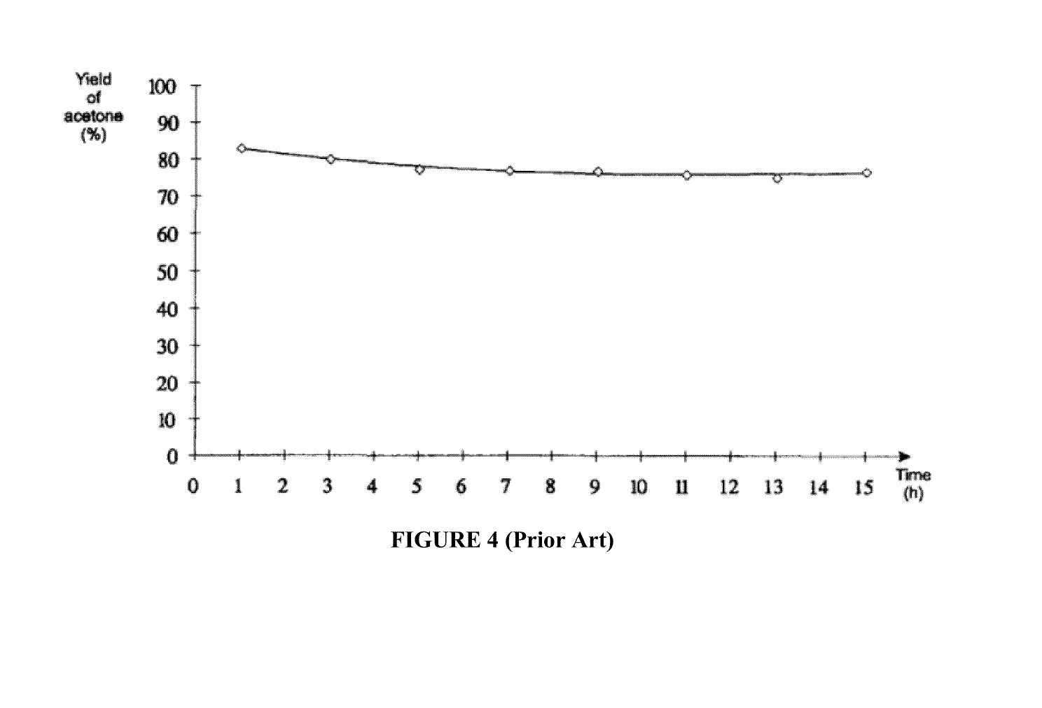

Catalyst performance may be evaluated based on acid conversion and ketone yield under conditions such as temperature, pressure (partial pressure of acids) and space velocity. Another factor on process catalyst application is its stability or longevity under process conditions. For example, the long-term performance study of 20% CeO₂—Al₂O₃ catalyst on acetic acid ketonization is shown in FIG. 4. The CeO₂ based catalyst tends to be highly stable for lower acid ketonization; however, stability for heavy acid ketonization needs to be evaluated, as coking with large molecules is a common observation in catalytic hydrocarbon processing.

Catalyst deactivation, regeneration or replacement is a significant factor for process operation and economics. If the catalyst requires regeneration with high frequency, a cheaper catalyst such as MgO/CaO is more attractive.

Single Pass and Recycle Process.

A continuous ketonization process of MCFAs may be designed depending on the catalyst performance. If the catalyst needs regeneration with high frequency, a moving bed or fluid bed reactor is used. For a relatively stable catalyst, a fixed bed reactor is used. Temperature, pressure, and space velocity (or space time) are three major design factors. In addition, the reactant co-feeding and product separation are also important considerations.

Ketonization of carboxylic acids is favored under high temperature and low pressure as shown from thermodynamics and kinetics discussions. As mentioned, ketonization of MCFAs is only slightly exothermic, thus, an adiabatic reactor may be adopted. The feed stream may be preheated and heat integration may be implemented to cool down the hot effluent with the feed stream. Because ketonization is favored at low pressure, there are two options to achieve this condition. One is single-pass with dilution of feed acid with inert gas such as N₂; another one is to recycle some ketone product as diluents. In some cases, the system/process takes place at lower pressures than atmospheric (i.e., vacuum).

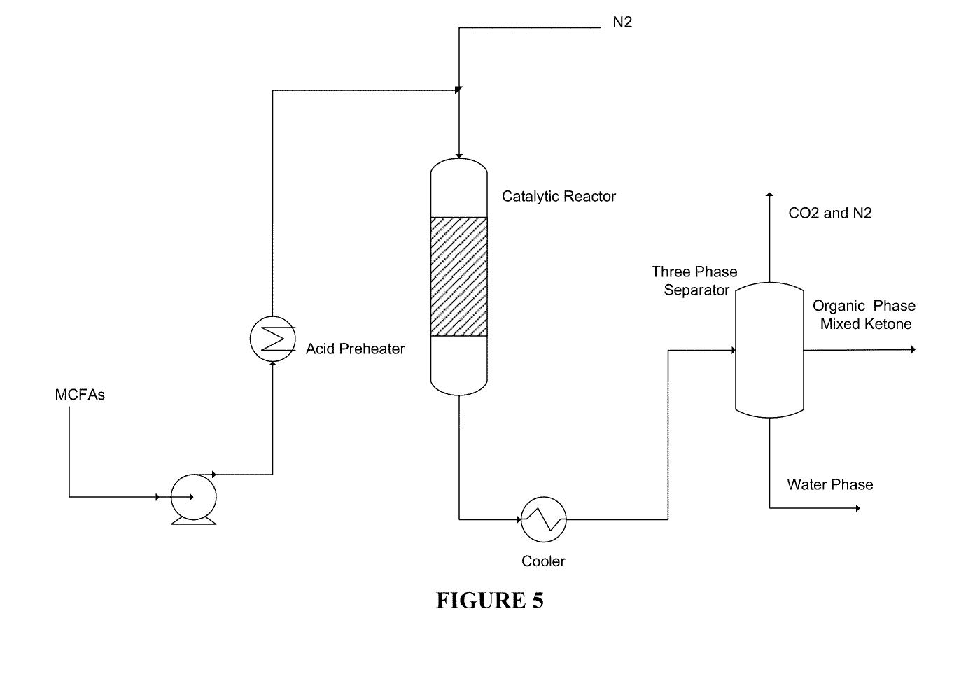

In an embodiment, as illustrated in FIG. 5, a single pass system is shown. The mixed acid feed is preheated to 300-400° C., then co-fed with N₂ into the fixed bed reactor packed with selected metal oxide catalyst. The reactor outflow is cooled down before flashing into a three-phase liquid-vapor separator. The vapor phase (comprising N₂, CO₂ and minimal other vapor side products) is vented and may be sent to a gas separation device such as a gas membrane, to separate the CO₂ from the N₂ so that the N₂ may be recycled. The water is separated from the bottom while the organic phase ketone product may be stored or transferred to the next process.

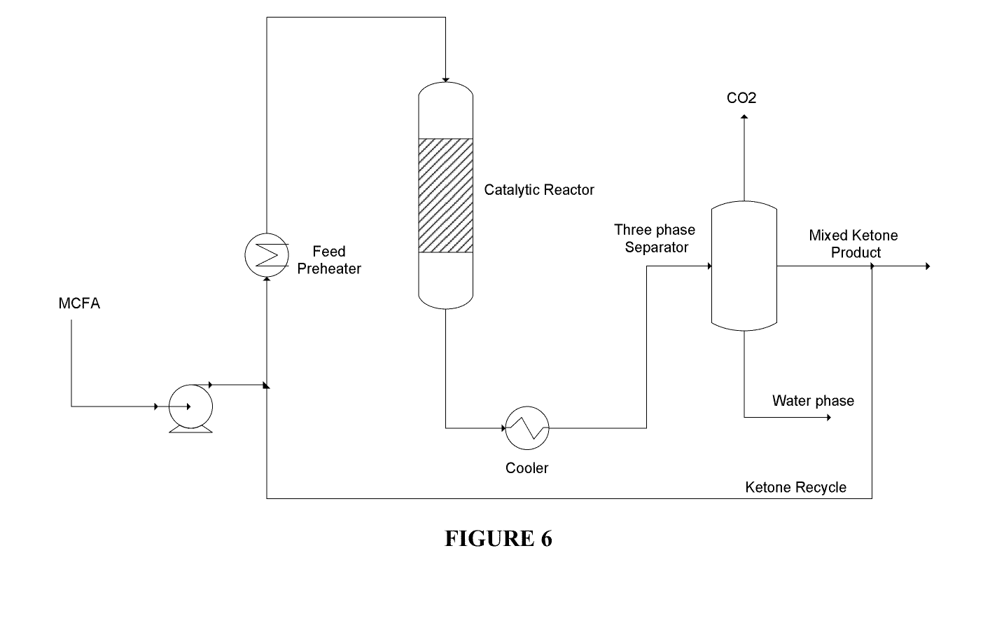

In an embodiment, as illustrated in FIG. 6, a system with ketone recycle is shown. The MCFA feed is mixed with some recycled ketone product, and preheated to 300-400° C. before being fed to the fixed bed reactor packed with the metal oxide catalyst. The reactor outflow is cooled down before being sent to a three phase separator. The vapor phase (comprising mostly CO₂) is vented, while the water phase may be removed from the bottom. After some of the organic ketone phase flow is recycled to mix with the acid feed, the rest of the ketone product may be stored or transferred to the next process.

Ketone Hydrodeoxygenation (HDO).

The mixed ketone product from MCFA ketonization comprises long-chain ketones, which may be valuable chemical products as well as fuel additives as oxygenates. In this disclosure, the mixed ketones are further processed to produce on-specification drop-in fuels, e.g., they are chemically upgraded to reduce the oxygen content.

The oxygen content has been reduced significantly from MCFA to ketones through the ketonization process by removing CO₂ and water byproducts. For example, heptanoic acid has 24.6 wt % oxygen compared to 8 wt % oxygen in 8-tridecanone (from two heptanoic acids ketonization). Although there is no specification requirement on oxygen level in the fuel product and oxygenates may be used directly as fuels or blending stock, e.g., ethanol and FAME (fatty acid methyl ester), the mixed ketones may need to be further processed to meet other specific requirements as drop-in fuels, such as, volatility, flash point, freeze point, cloud point, viscosity, octane number, cetane number, and thermal or oxidative stability.

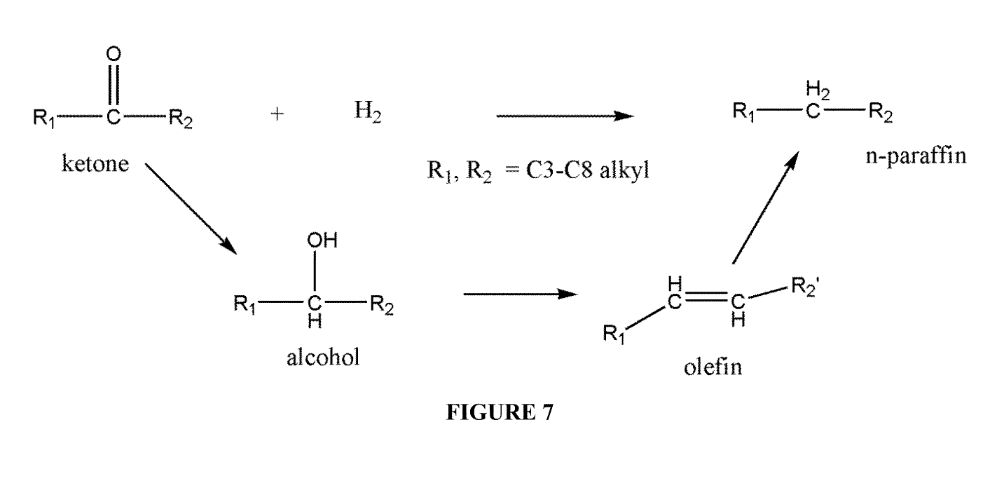

FIG. 7 illustrates a mechanism/pathway to chemically upgrade the mixed ketones to make hydrocarbons which are chemically identical or similar to petroleum refinery products. To reduce oxygen from the long-chain ketones, hydrogen is used to combine with oxygen and form water. Linear C7-C17 n-paraffins are the hydrocarbon products. The mechanism as shown in FIG. 7 may be promoted by a hydrotreating catalyst or a hydrogenation catalyst to convert ketones to alcohols, then to dehydrate to olefins, and lastly to hydrogenate the olefins to paraffins. Alternatively, each of these steps as shown in FIG. 7 may occur separately with different catalysts either in several catalytic reactors on a stacked-bed catalytic reactor. This process may also be applied to produce intermediate products such as medium-chain secondary alcohols and internal olefins.

In some cases, when high yield of diesel-range fuels are desired, the shorter long-chain ketones, such as C7 and C8 may be elongated using appropriate oligomerization catalysts, such as those that would elongate olefins or catalysts that may cause ketones to undergo aldol condensation. Nonetheless, the one-step hydrogenolysis of ketones to paraffins through hydrodeoxygenation (HDO) is more economically attractive.

Hydrotreating such as hydrodeoxygenation (HDO) is the catalytic reaction of H₂ with heteroatoms in oxygenates to form water and hydrocarbons. The H₂ consumption and severity of the operation to achieve high conversion of HDO is dependent on the content and O-type compounds in the feed. In comparison to complex mixtures of bio-oils or bio-crudes obtained from pyrolysis, mixed ketones derived from ketonization of MCFAs are relatively simple compounds with only one ketone functionality that are easily hydrotreated. Aliphatic ketones are among the easiest compounds to be hydrotreated with lower activation energy and reaction temperature compared to other complex oxygenates like phenols and furans.

The catalyst systems for hydrodeoxygenation or hydrogenolysis of oxygen functions such as ketones, aldehydes, alcohols, ethers or esters include hydrogenation catalysts of metal oxides or Group VIII transition metals, e.g., copper chromite (CuO.CuCr₂O₈) and palladium on carbon (Pd/C). Reaction conditions include a temperature higher than 250° C. and a pressure more than 3000 psig (since ketones are normally converted to alcohols only under mild conditions). Many side products may be produced under such conditions, and some alcohols may be resistant to hydrogenolysis.

Some homogenous catalysts containing iridium or rhodium component and halogen component have been also reported for hydrotreating. However, batch operation as well as separation of homogeneous catalyst makes it difficult to apply on large scale fuel production.

Other catalysts may be used for hydrogenolysis of C—O and C═O bonds in organic substrates with mixed aromatic and aliphatic alkyl groups, such as a catalyst that contains at least one hydrogenating metal Group VIII (Fe, Co, Ni, Ru, Rh, Pd, Os, Ir and Pt) and/or IB (Cu, Ag, and Au) impregnated on solid acid supports, e.g., zeolites such as ZSM-5, mordenite, zeolite Y and Beta zeolite. The bifunctional Pd/zeolite catalyst is able to convert 1-phenylethanol to ethylbenzene under ambient condition (30° C. and 1 bar). In contrast, commercial 5% Pd/C catalyst gives no significant conversion without addition of liquid sulfuric acid.

Hydrotreating catalysts in petroleum refining process may also be utilized, such as cobalt plus molybdenum or nickel plus molybdenum in the sulfide forms, impregnated on an alumina base (CoMo/Al₂O₃ or NiMo/Al₂O₃). The temperatures employed are in the range of 300° C. to 350° C. and the hydrogen pressures are about 500 to 1000 psi. More severe conditions may be applied for deep desulfurization of high S content petroleum feedstock; however, high severity will drive hydrocracking reactions to occur to a certain degree. During hydrotreating, hydrodesulfurization (HDS), Hydrodinitrogenation (HDN), Hydrodeoxygenation (HDO), hydrodemetallization (HDM), and hydrogenation (HYD) occur simultaneously depending on the heteratom content of the feedstock and process conditions. Thus, metal sulfide catalysts like CoMo/Al₂O₃ and NiMo/Al₂O₃ may be used for HDO for bio-oil upgrading and coal-to-liquid processes.

While traditional precious metals (Pd and Pt) or Ni hydrotreating catalysts on alumina or silica supports suffer the drawbacks of being easily poisoned by S-containing compounds and deactivation due to carbonaceous deposits, a catalyst based on molybdenum or tungsten disulfide was developed for hydrotreating of coal derived liquids. However, the metal sulfide catalyst will have its own drawback when used for hydrotreating of oxygenate feedstock which does not usually contain sulfur compounds. Catalysts comprising active sulfided metal centers tend to be relatively unstable as the metal sulfides breakdown to their oxide forms in the environment of oxygenates and steam.

Other methods for the hydrodeoxygenation of oxygenate feedstock with sulfided catalysts, which have the limited build-up of carbonaceous deposits and stabilization of metal sulfide catalyst centers. The catalyst composition comprises a porous carrier (alumina with ˜0.001-1 wt % phosphorous and ˜0.001-1 wt % silicon) and 1-20 wt % of active sulfide metals of Group VIB (e.g., Mo) and Group VIII (e.g., Ni). The oxygenate feedstock comprises at least 10 wt % of glyceride. The hydrogenation conditions comprise a temperature in the range of 250-400° C., hydrogen partial pressure in the range of 10-220 bar, and a liquid hourly space velocity (LHSV) in the range of 0.1-10 hr⁻¹. In some cases, in order to stabilize the metal sulfide active center during the process, hydrogen sulfide (H₂S) or a precursor (e.g., CS₂) is present in the hydrogen gas in the amount of 10-10000 ppm. Nonetheless, if the feedstock contains a sufficient amount of sulfur, supplemental sulfur does not need to be added.

Ketone Hydrogenolysis Process.

Direct hydrogenolysis or hydrodeoxygenation of ketones is a one-step process to upgrade the ketone to hydrocarbon products. As mentioned, aliphatic ketones are among the easiest oxygenates compounds for HDO under mild conditions. In some embodiments, two types of catalysts (designated herein as type A and type B) are applied to upgrade the ketones to hydrocarbons. Type A is Group VIII metal (e.g., Pd, Ni, Ru) impregnated in zeolites (e.g., ZSM-5); Type B is Group VIB (e.g., Mo) and Group VIII (e.g., Co or Ni) in the sulfided form supported on the Alumina. Table 4 lists the major properties of these two catalysts.

In addition to the catalysts discussed above, other hydrotreating catalysts may also be used for the ketone hydrogenolysis process as known to one skilled in the art.

TABLE 4
List of major properties for catalysts
used to upgrade ketones to hydrocarbons

| Catalyst Type | Type A | Type B |
|---|---|---|
| active center | Pd or Ni metal | Sulfided Co/Mo/Ni |
| support | Zeolites | Alumina |
| temperature | Low | High |
| pressure | Low | High |
| deactivation | Carbonaceous deposits | Sulfides change to oxides |
| regeneration | Coke burning | Sulfur treating |

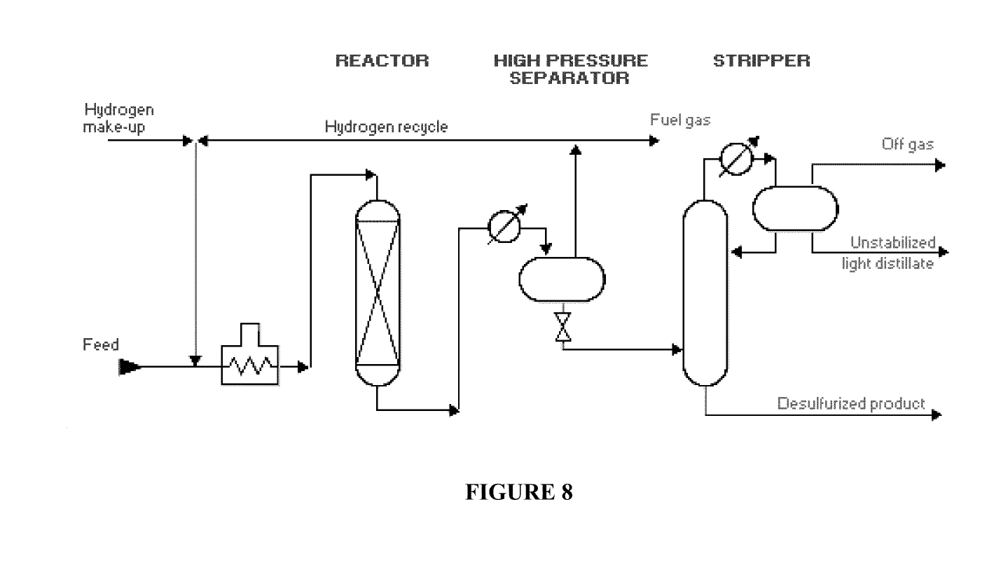

In an embodiment, this process is illustrated in FIG. 8 as a continuous process. The process starts with the co-feeding of the preheated ketones with hydrogen gas into the reactor with either type A or type B catalyst under set conditions of temperature and pressure. The effluent out of the reactor is cooled before it goes to the knockout drum, in which unreacted H₂ is separated and recycled. The water phase in the liquid product is removed as waste and the organic phase liquid hydrocarbon product is collected or transferred to next stage for further refining to transportation fuels.

Some metrics are discussed on the process performance and economics:
(1) Conversion. The oxygen content in the mixed ketone of C7-C17 is expected in the range of 5-15%. Unlike hydrodesulfurization (HDS) to make ultra low sulfur fuel (ppm levels of S), oxygen level of <1% in the final hydrocarbons is acceptable. Thus, 90-95% conversion of HDO is pursued in this process.
(2) Selectivity or yield. During hydrotreating especially HT/HP process using CoMo/Al₂O₃ or NiMo/Al₂O₃, some degree of cracking will occur and decrease the average molecular weight of hydrocarbon mixture. Optimized temperature/pressure conditions are needed to minimize hydrocracking during the hydroprocessing. Coking or other carbonaceous deposits on catalysts may compromise the total hydrocarbon yield, thus they need to be controlled as well.
(3) Catalyst cost. Either Pd/Zeolite or CoMo/Alumina catalyst may be regenerated. The catalyst cost for the process is not significant. A catalyst stability test for regeneration frequency is important to compare the economics.
(4) Process temperature, pressure. Since hydrotreating using CoMo or NiMo catalysts requires high temperature and high pressure, the engineering design on the reactor operation tend to be more demanding. In contrast, low temperature low pressure hydrotreating with Pd/zeolite is more attractive and it may achieve the same level of conversion and selectivity with stabilized catalyst.

Catalytic Reforming.

As discussed above, MCFAs are upgraded through ketonization to produce longer-chain ketones in the range of C7-C17. The subsequent hydrodeoxygenation step produces a mixture of hydrocarbons which comprises mostly linear n-paraffins in the range of C7-C17. The C10+ linear paraffins are hydrocarbons in the diesel range. With high cetane number as normal paraffins, a mixture of C10-C17 paraffins meet specifications on density, viscosity, cloud point and other cold-flow properties or may need to undergo minor refinement, and thus such mixture may be used as drop-in diesel fuel.

In an embodiment, a distillation step follows to fractionate the HDO product into a heavy fraction of C10+ used as diesel fuel, and a light fraction of comprising hydrocarbons shorter than C10. This light fraction, in some cases, is similar to the naphtha fraction from petroleum atmospheric distillation. In an embodiment, the mixture of C7-C10 normal paraffins has low octane number (RON of n-heptane is 0, and RON of C8+ is <0) and in these cases, the C7-C10 ketones may be separated so that they may undergo oligomerization (either by converting them to olefins and then oligomerizing such olefins, or by aldol condensation of the ketones) followed by hydrotreating. Such oligomerization would move the C7 to C10 ketones into the diesel range. Alternatively, catalytic reforming technology may be applied to the C7-C10 normal paraffins to increase its octane number, and the reformate product with high octane number may be used as blending stock for motor gasoline as well as aviation gasoline.

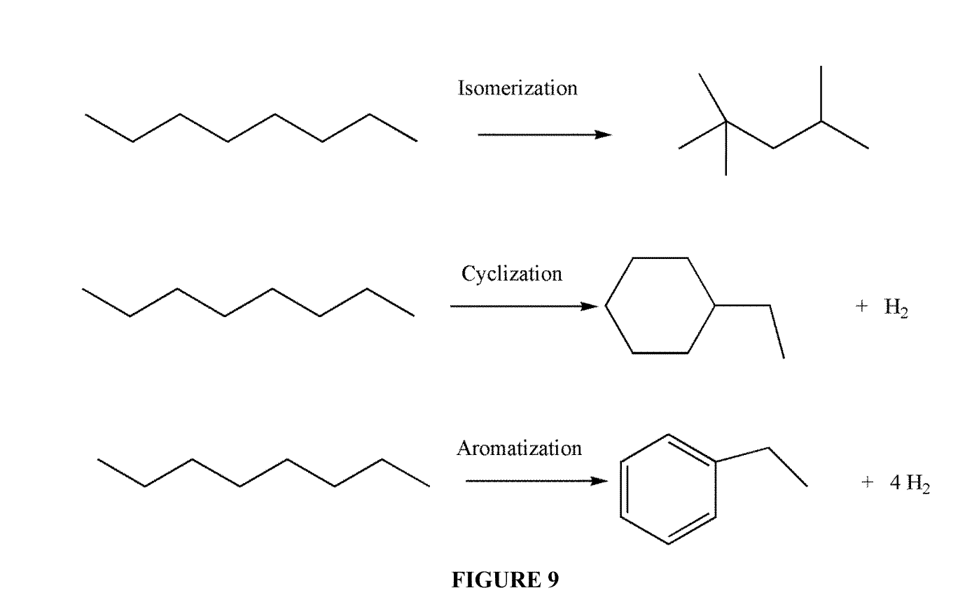

In an embodiment, catalytic reforming is conducted in the presence of hydrogen over hydrogenation-dehydrogenation catalysts supported on alumina or silica-alumina. Catalytic reformer feeds are saturated naphtha like C5-C10 range paraffins. Depending on the catalyst and condition, several types of chemical reactions occur in the reforming reactor. Paraffins are isomerized to branched chains and hydrocyclized to naphthenes, and naphthenes are converted to aromatics. For example, n-octane may be reformed to iso-octane, ethyl-cyclohexane, or ethylbenzene (FIG. 9). Overall, the reforming reactions are endothermic. The resulting product stream (reformate) from catalytic reforming has a RON ranging from 96 to 102. Thus, the light fraction of C7-C10 n-parafins through catalytic reforming provides a high octane number aromatic blending components, which may be used in both motor gasoline and aviation gasoline. In addition, the dehydrogenation reaction which converts paraffins to unsaturated aromatics produces hydrogen, which may be used as makeup hydrogen for ketone HDO process.

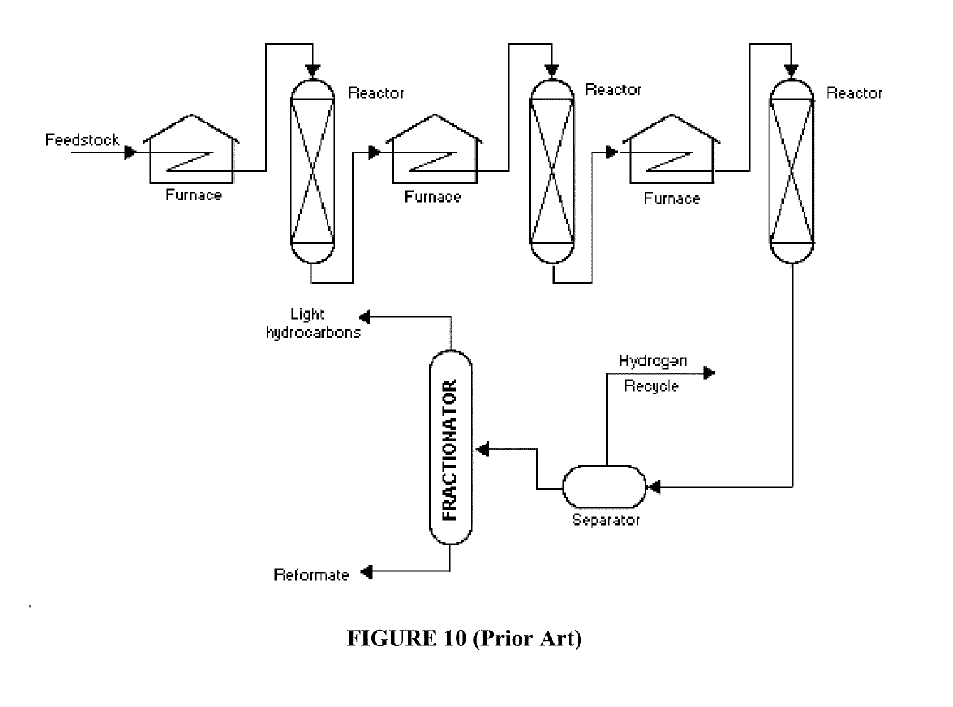

In another embodiment, the catalytic reforming process is carried out by feeding the naphtha (saturated paraffins) and hydrogen mixture through a pre-heater where the mixture temperature is increased to 450-520° C., and is passed through fixed-bed catalytic reactors at hydrogen pressure of 100 to 1000 psi. In some cases, two or more reactors are used in series, and re-heater(s) are used between reactors to compensate for the endothermic reactions. The product effluent stream is cooled and sent to high-pressure separator where hydrogen-rich gas is recovered for recycle and a split of extra hydrogen production is available for other uses. The liquid product (reformate) is stabilized (by removal of light ends) and used directly in gasoline or extracted for aromatic blending stocks for aviation gasoline. Such a process is illustrated in FIG. 10.

Hydroisomerization and Hydrocracking of Heavy n-Paraffins.

In an embodiment, from the HDO product of mixed normal paraffins in the range of C7-C17, diesel fuel and high octane number gasoline is produced through distillation and catalytic reforming of light fraction. It is more demanding to make aviation fuels such as Jet A or JP8, as the jet fuel has more stringent specification requirements than gasoline and diesel. The kerosene-type jet fuel has hydrocarbons in the range of C9-C16 (average in C11-C12 range), and has a boiling point ranging from about 150° C. to about 300° C. The normal paraffinic kerosene normally has very high freeze point, which does not meet the freeze point specification (<−58° C.). In addition, a smooth distillation profile (a minimum slope) on fuel volatility specification is demanded for turbine engine operation. Thus, iso-paraffinic kerosene (IPK) is a typical type of jet fuel. The energy content and combustion quality of jet fuel also demand some level of aromatic content. Currently a minimum 8% and a maximum of 25% aromatics are required for JP8.

Although paraffin hydroisomerization has been extensively used in petroleum refinery, there is a significant difference in the isomerization of C4-C7 and heavier paraffins, which is reflected in the classification of hydroisomerization. The classification reflects differences in catalyst selection, application objectives, and technology. The isomerization of heavier paraffins may be performed by hydrocracking or hydroisomerization, and the decision of selecting one over another is related to the desirability of shifting the carbon number distribution.

The heavy paraffinic materials are susceptible to cracking, and hydroisomerization catalysts are also potential hydrocracking catalysts. Thus, the catalysts and process conditions have been optimized to increase isomerization while suppressing cracking. In an embodiment, hydroisomerization of long-chain n-paraffins is carried out over bifunctional catalysts, which have the first function of hydrogenation-dehydrogenation and the second function of skeletal isomerization of olefins. Catalysts that have a high degree of hydrogenation activity and a low degree of acidity are preferable for maximizing hydroisomerization and suppressing hydrocracking. Such catalysts include noble metals (like Pt and Pd) on low acidity supports (such as zeolites). In some cases, Pt/CaY zeolite catalyst in the presence of hydrogen is used for isomerization. In other cases, zeolites with medium pore size and low acidity such as ZSM-11 and SAPO-11 are utilized to produce high isomerization selectivity.

In other embodiments, medium chain fatty acids in the range of C4-C9 produced through controlled mixed-acid fermentation in the upstream are upgraded to transportation hydrocarbon fuels effectively and efficiently through three major steps:
(1) Hydrogenation of MCFAs to primary alcohols, which may occur in either by directly hydrogenating the acids or by converting the acids to esters and then hydrogenolyzing the esters, to produce the alcohols. Esterification may occur by reacting alcohol with the acids, or by reacting olefins with the acids. Nickel and copper based catalysts are adequate for the hydrogenation process. Such reaction results in primary alcohols ranging from C4 to C9.
(2) Dehydration of the alcohols followed by dimerization or trimerization of the resulting olefins, which are reactions that occur in the same reactor and over the same catalyst, such as but not limited to, β zeolites and other zeolite-type catalysts. This reaction results in the production of n-paraffins of size ranging from C8 to C18.
(3) Refining of paraffinic dimerization/trimerization product to transportation fuels. Various processes and systems may be configured according to the desired products as known to one skilled in the art. The on-specification kerosene-type jet fuel or diesel fuel, gasoline or aviation gasoline may be produced from the dimerization/trimerization product specifically by selecting the appropriate processes, such as but not limited to, hydroisomerization, hydrocracking, catalytic reforming, and fractionation/distillation.

Integrated Process for Transportation Fuel Production.

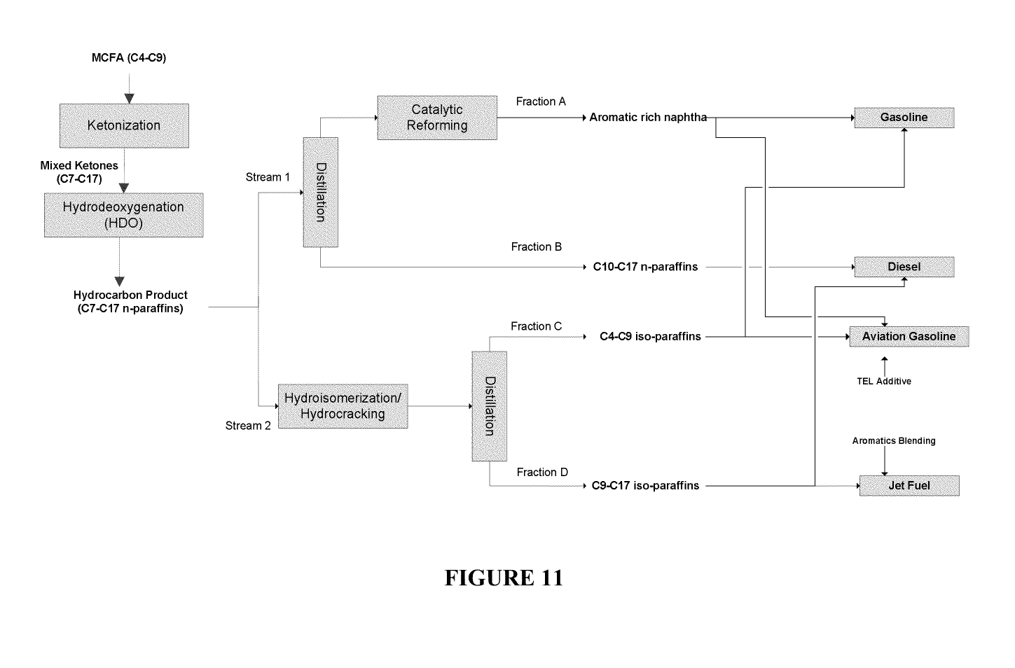

In some embodiments, MCFAs are ketonized and hydrogenated to produce hydrocarbons, comprising mainly C7-C17 n-paraffins; such hydrocarbons are then further processed into transportation fuels such as gasoline, diesel, jet fuel and aviation gasoline. Production of the four types of fuels is illustrated in FIG. 11. In some embodiments, following ketonization of the MCFA feed and hydrodeoxygenation (HDO) of the mixed ketones, the crude hydrocarbon stream is sent to two different processes. Stream 1 goes directly to a distillation tower, where the heavy fraction B of mostly C10+ normal paraffins is the major components for diesel fuel, and the light fraction of mostly C7-C9 is upgraded through catalytic reforming and the reformate (Fraction A) is aromatic rich and is a blending stock for high octane gasoline. Stream 2 is upgraded through hydroisomerization or hydrocracking process and the resulting iso-paraffin stream goes to fractionation, where the heavy iso-paraffin fraction D is the major component of jet fuel, and the light fraction C is a blending stock for either motor gasoline or aviation gasoline. Depending on the target market or desired use, the process may be adjusted to produce various products as known to one skilled in the art.

In an embodiment, for jet fuel production, all the HDO product may go through the hydroisomerization/hydrocracking process while the catalyst and conditions are selected to maximize the hydroisomerization selectivity. The iso-paraffinic kerosene (Fraction D) after distillation may be obtained with high yield (>50%) and used as the major component in jet fuel, and the byproducts of jet fuel production (Fraction C) are good gasoline blend stock. To meet its specifications either for Jet A or JP8, small quantities of petroleum-based heavy aromatics such as hydrotreated light cycle oil (LCO) and other additives such as antioxidants can be blended.

In another embodiment, to make high octane Aviation gasoline (AVGAS), the light fraction of some HDO product distillation needs to be catalytically reformed (Fraction A) to make the aromatics with very high octane number (RON>100). The heavy fraction from distillation combined with more HDO product may go through the hydroisomerization or hydrocracking process, where the catalyst and condition are selected to maximize the C5-C9 isoparaffins in the effluent stream. The light Fraction C is high octane gasoline with octane number around 80-90. AVGAS may be prepared by blending the major component of C5-C9 isoparaffinic gasoline, some aromatic rich fraction A, and certain amount of Tetraethyllead (TEL) depending on the AVGAS grade specifications.

In yet a further embodiment, to produce Diesel from the HDO product, the n-paraffinic kerosene (high cetane number) is used directly as diesel blend stock after the light fraction of mostly C7 to C9 paraffins is removed to meet the flash point specification (minimum 55° C.). The cold-flow properties such as cloud point, pour point and freeze point are important for diesel fuel, which may be problematic for heavy linear paraffins in the range C15-C22, which are not significantly present in the HDO product of this process. Further, a minor degree of hydroisomerization applied to the HDO product from this process may optimize the kerosene type diesel fuel to meet its specifications.

In an embodiment, the light fraction stream from diesel production is upgraded through catalytic reforming to make high octane gasoline. In another embodiment, the light fraction stream from diesel production combined with light fraction from jet fuel production can be upgraded through catalytic reforming to make high octane gasoline.

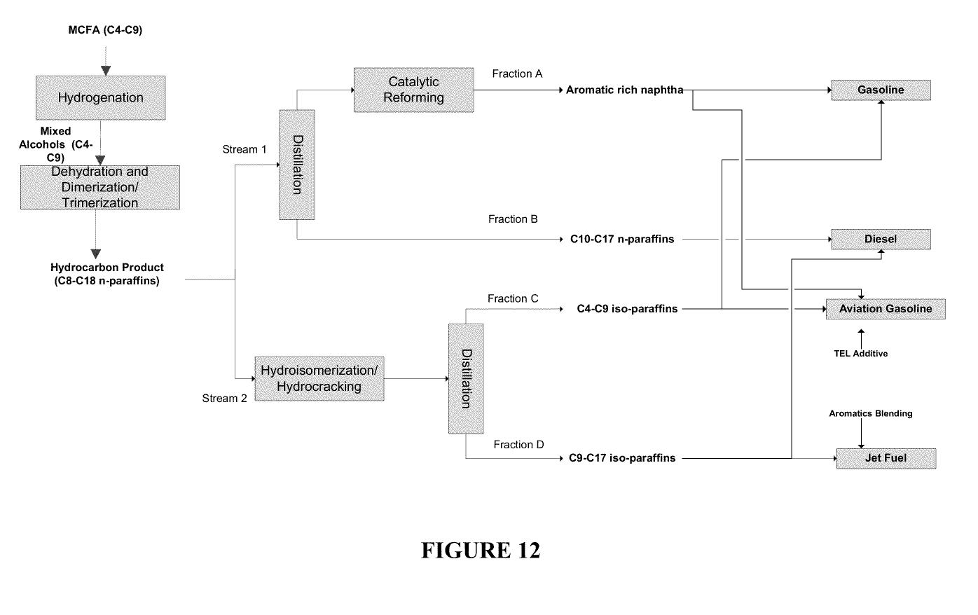

In some embodiments, MCFAs are hydrogenated to form primary alcohols are dehydrated and dimerized or trimerized to form a product, comprising mainly C8-C18 n-paraffins; such hydrocarbons are then further processed into transportation fuels such as gasoline, diesel, jet fuel and aviation gasoline. Production of the four types of fuels is illustrated in FIG. 12. Description of the downstream conversion of the n-paraffins in FIG. 12 is similar to the description of the downstream conversion of FIG. 11 described above.

Advantages.

The system and method as disclosed herein provide the details for converting medium-chain fatty acids (e.g., recovered from mixed culture fermentation) into hydrocarbon biofuels. Medium-chain fatty acids are selectively produced from fermentation. For shorter chain fatty acids (acetic and propionic), extensive oligomerization to elongate the carbon chain is necessary and production of diesel is difficult since the oligomerization reaction has difficulty generating molecules that are long enough to meet the diesel specifications. In addition, some of the catalysts for the oligomerization process may be easily poisoned by sulfur and nitrogen impurities. The process of this disclosure, on the other hand, starts with MCFAs and is able to obtain the proper carbon length from the ketonization step without the need of oligomerization, thus making it easy to produce diesel and jet fuel. Gasoline may also be produced from this process, but it is not recommended for exclusive gasoline production as one of the benefits of using ketonization is to elongate the hydrocarbon molecules for diesel and jet fuel production. The gasoline fraction, which may be a blend stock, may be a byproduct during the jet fuel and diesel production. To make gasoline as the major product, the HDO product may go through the hydrocracking process to produce mostly isoparaffins in the range of C4-C9. Some paraffins may go through catalytic reforming to increase the gasoline octane number. In another alternative process, the medium chain fatty acids are converted to n-paraffins through decarboxylation (removal of CO₂ from carboxylic acid) or hydrogenation of acid or its ester followed by dehydration of the resulting alcohols. The low octane n-paraffins in the range of C4-C10 may be catalytically reformed to make high octane gasoline.

The process of ketonization followed by hydrogenolysis to convert MCFAs into hydrocarbons produces higher yields compared to processes that used short-chain fatty acids because oxygen, which needs to be removed, is a smaller percentage of the medium-chain fatty acids compared to the short-chain fatty acids. The process is not limited by sulfur or nitrogen poisons because the first step after ketonization is hydrotreating to hydrodeoxygenate the ketones, which removes sulfur (S) and nitrogen (N) impurities.

The process of hydrogenating the MCFAs to convert them to alcohols, followed by dehydration and dimerization/trimerization to convert them into hydrocarbons uses more hydrogen than the process that employs ketonization, but results in higher yields because it avoids the loss of CO₂ that occurs during the ketonization process.

### Examples

### Refining of n-Paraffins to Transportation Fuels

Hydrotreating of mixed long-chain ketones will produce a mixed hydrocarbon stream, which comprises of mostly C7-C17 normal paraffins with minimal amounts of small paraffin and isoparaffin molecules from hydrocracking and from the conversion of some of the iso-acids, such as isobutyric and isovaleric acids, produced in the fermentation. The hydrocarbon stream is similar to middle distillates of paraffinic petroleum except the carbon is biomass sourced, and can be blended into similar petroleum products as renewable components.

It is of interest to make drop-in fuel products from renewable resources as it requires no engine modification and no infrastructure change and also minimize the need for blending with petroleum products. To make on-specification fuel products, the product from HDO of mixed ketones needs extra refining processes similar to petroleum refining, such as, distillation, reforming, isomerization, cracking, and fuel product formulation.

Specifically, the transportation fuels like gasoline (motor gasoline and aviation gasoline), kerosene-type jet fuel, and diesel, are the targeted products for the market; therefore, the bio-refining process must be designed to make each type of fuel and meet specification requirements. The properties and specifications of the fuel products are discussed and summarized in Table 5.

TABLE 5
Selected typical properties for desired products

| | Gasoline | Jet Fuel | Diesel |
|---|---|---|---|
| Product | | | |
| Boiling Point | 30-210 | 140-300 | 150-380 |
| Range (° C.) | | | |
| Carbon Range | C4-C10 | C8-C17 | C10-C22 |
| Density (kg/m³) | 720-780 | 775-840 | 820-860 |
| Vapor Pressure | 35-70 | <21 | — |
| (kPa) | | | |
| Flash Point | <−40 | >38 | >55 |
| (° C.) | | | |
| Freezing Point | — | <−40 | ~0 (cloud point) |
| (° C.) | | | |
| Hydrocarbon | | | |
| Compositions | | | |
| Paraffins | — | >75% | >70% |
| Olefins | <20% | — | — |
| Aromatics | 30-40 vol % | 8-25 vol % | <30 wt % |

Gasoline (or specifically motor-gasoline) is the oldest transportation fuel and is used in the spark-ignition engine or Otto engine. Its specifications are dependent on location and season and have technical, environmental, and political origins. Guidelines for motor-gasoline specifications in the United States are published in the ASTM D 4814 standard test method. Some selected properties are described here for relative importance to our crude hydrocarbon product refining.
(1) Octane number. The octane number or anti-knock index of gasoline is characteristic of the auto-ignition resistance during the fuel compression in the engine. The correlation of chemical compositions in the fuel to the octane number is well established in that aromatics and isoparaffins are octane boosters while linear paraffins have very low octane number.
(2) Volatility. The volatility is characterized by the distillation profile and vapor pressure of gasoline. They fluctuate on different seasons and different locations. Thus the refining process is always somewhat flexible to adjust the distillation cut for different market use.
(3) Fuel stability. The storage stability of a fuel is dependent on its oxidation stability. The auto-oxidation of hydrocarbons takes place through free radical mechanism when high olefin content is present, and results in fuel degradation to oxygenates, which can form heavier compounds or gum, which drags the engine performance. Thus, there is maximum allowable alkene level in the gasoline due to its high reactivity to form gums. In addition, fuel additives such as oxidation inhibitors and radical scavengers are added.
(4) Aromatic content. There is a decreasing trend in the aromatic content in gasoline; however, the decreasing aromatic level needs be compensated by increasing high octane paraffins, which will increase the refining cost. The aromatic level will likely be kept at 30% level, while the maximum benzene content will be less than 1% due to environmental and safety concerns.
(5) Oxygenate content. Although the high octane oxygenates can be attractive for fuel blending, the amount and species of oxygenated compounds in gasoline are regulated. Except for ethanol used in large quantities as the major blending stock for new gasoline, other oxygenates are restricted as some additives. There are maximum allowed oxygenate content (currently 2.7%), and most are listed as ethers and light alcohols (other than ethanol).

Aviation-gasoline is used as fuel for spark-ignition engines on aircrafts. It is similar to motor-gasoline, but it has more stringent requirements due to the performance demands placed on aircraft engines. The requirements for AVGAS are published in ASTM D910 standard method and some are listed in the Table 6. Compared to motor gasoline, AVGAS has freezing point of −58° C. It has less heavy end with the final boiling point (FBP) of max 170° C. (FBP of gasoline is ˜200° C.). In order to achieve high octane number or RON/MON, Tetraethyllead (TEL) is allowed into AVGAS, which is prohibited in motor gasoline in the United State and most of the world.

TABLE 6
Selected aviation gasoline (AVGAS) specifications
for different grades in the U.S.

| | | | | |
|---|---|---|---|---|
| AVGAS Grade | 80 | 91 | 100 | 100LL |
| Peformance number, min | — | — | 130 | 130 |
| RON, min | 87 | 98 | 106.5 | 106.5 |
| MON, min | 80 | 91 | 99.5 | 99.5 |
| Vapor pressure at 37.8° C. (kPa) | 38-49 | 38-49 | 38-49 | 38-49 |
| Freezing point (° C.) | −58 | −58 | −58 | −58 |
| Net Heat of combustion (MJ/Kg), min | 43.5 | 43.5 | 43.5 | 43.5 |

Aviation turbine fuel, or jet fuel, has displaced aviation-gasoline as the main fuel type for aircraft, as most aircrafts nowadays are equipped with turbine engines instead of spark-ignition engines. Jet fuel is a kerosene-range fuel, and it requires very different fuel properties than AVGAS. The energy content and combustion quality of the fuel are key performance properties. In contrast to gasoline with large variation of specification requirements, the specifications for jet fuel are of little change internationally. The civilian fuel Jet A or A-1 and military fuel JP-8 are not significantly different on chemical contents and other specifications. The US uses Jet A for national travel, which is defined by the ASTM D 1655 standard. JP-8 specifications are defined in publication MIL-DTL-83133H. Some major properties of Jet fuel are listed in table 7.

TABLE 7
Selected specifications for civilian
and military aviation turbine fuels.

| | Jet A | JP-8 |
|---|---|---|
| Property | | |
| Net Heat Combustion (MJ Kg⁻¹), min | 42.8 | 42.8 |
| Density at 15° C. (Kg/m³) | 775-840 | 775-840 |
| Freezing point (° C.), max | −40 | −47 |
| Flash point (° C.), min | 38 | 38 |
| Viscosity at −20° C. (cSt), max | 8 | 8 |
| Final Boiling Point (° C.), max | 300 | 300 |
| Composition, max. | | |
| Aromatic content (vol %) | 25 | 25 |
| Naphthalene content (vol %) | 3 | 3 |
| Sulfur content (mass %) | 0.3 | 0.3 |
| Thiol content (mass %) | 0.003 | 0.002 |
| Acid content (mg KOH/g) | 0.1 | 0.015 |

Diesel fuel is used in compression-ignition engines (diesel engines), and is generally fuel with boiling point in the C10-C22 hydrocarbon range. Compression-ignition engines are robust and can operate reliably with heavy fuels of quite varied composition. The guideline for diesel fuel specification in the United States is reported in ASTM D 975 standard. A few selected properties of diesel fuel whose specifications need to be met in relation to our product from HDO are as follows:
(1) Cetane number. The cetane number is a measure of the compression-ignition delay of fuel. Different from the octane number, linear long-chain paraffins have higher cetane number than their branched isomers (n-hexadecane has a value of 100). A minimum 40-50 of cetane number is required in most of the world.
(2) Density and viscosity. The density and viscosity of the fuel determine the performance of the injection system. The progressive narrowing of both the density and viscosity range for diesel fuel specification is intentional to maintain engine performance and control emissions.
(3) Aromatic content. Like jet fuel, the diesel fuel specifications differentiate total aromatic content and polynuclear aromatics (naphthalene). The total aromatic content determines the flame temperature during combustion. A high aromatic content causes high particulate matter (PM) and NOx emissions. Similar to isoparaffins, the high octane aromatics have a low cetane number, which make aromatics not critical components in diesel fuels as opposed to in gasoline.
(4) Cold-flow properties. The region-specific cold-flow properties are very important in diesel specifications and tested as cloud point and cold filter plugging point. At low temperature, the high cetane components (linear paraffins) tend to crystallize out of solution as wax, which can block the fuel filter and fuel lines. Thus, some degree of isomerization is common practice to improve the kerosene-type diesel cold-flow properties.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide exemplary, procedural or other details supplementary to those set forth herein.

We claim:

1. A method for producing hydrocarbon fuels from biomass, the method comprising:
converting at least a portion of the biomass into medium-chain fatty acids or carboxylic acids ranging from C4 to C9;
reacting at least a portion of the medium-chain fatty acids or carboxylic acids in a ketonization reactor to produce a ketonization product; and
reacting at least a portion of the ketonization product in a hydrodeoxygenation reactor to remove substantially all oxygen and produce a hydrodeoxygenation product comprising n-paraffins.

2. The method of claim 1 wherein said medium-chain fatty acids or carboxylic acids ranging from C4 to C9 are produced via controlled fermentation.

3. The method of claim 2 wherein said controlled fermentation comprises introducing a compound into said fermentation to effect chain elongation by microorganisms, wherein said compound is selected from the group consisting of ethanol, lactic acid, hydrogen and combinations thereof.

4. The method of claim 2 wherein said controlled fermentation comprises separating C2-C3 fatty acids or carboxylic acids from the fermentation product and recycling them to the fermentation for chain elongation.

5. The method of claim 3 or claim 4 wherein conversion of biomass to acids and chain elongation take place in separate reactors or separate series of reactors operated under different conditions.

6. The method of claim 1 wherein said ketonization takes place at a temperature in the range of from 200° C. to 600° C.

7. The method of claim 1 wherein said ketonization takes place at a temperature in the range of from 300° C. to 500° C.

8. The method of claim 1 wherein said ketonization takes place at a pressure in the range of from 0.1 atm to 10 atm.

9. The method of claim 1 wherein said ketonization utilizes a catalyst comprising oxides of cerium, oxides of manganese, oxides of calcium, oxides of magnesium, or combinations thereof.

10. The method of claim 1 wherein said ketonization produces a ketone yield of greater than 90%.

11. The method of claim 1 wherein said hydrodeoxygenation takes place at a temperature in the range of from 0° C. to 600° C.

12. The method of claim 1 wherein said hydrodeoxygenation takes place at a temperature in the range from 200° C. to 500° C.

13. The method of claim 1 wherein said hydrodeoxygenation takes place at a pressure in the range of from 0.1 to 250 atm.

14. The method of claim 1 wherein said hydrodeoxygenation utilizes a catalyst comprising cobalt, molybdenum, nickel, palladium, ruthenium, or combinations thereof.

15. The method of claim 1 comprising adding sulfur to stabilize said hydrodeoxygenation catalyst.

16. The method of claim 1 wherein said ketonization product comprises C7-C17 ketones.

17. The method of claim 1 wherein said hydrodeoxygenation product comprises C7-C17 n-paraffins.

18. The method of claim 1 wherein said hydrodeoxygenation product is further processed to produce transportation fuels or drop-in fuels.

19. The method of claim 1 wherein said hydrodeoxygenation product is further processed to produce diesel or jet fuel or both.

20. The method of claim 1 further comprising processing said hydrodeoxygenation product via hydroisomerization, hydrocracking, catalytic reforming, fractionation, distillation, or combinations thereof.

21. A method for producing hydrocarbon fuels from biomass, the method comprising:
converting at least a portion of the biomass into medium-chain fatty acids or carboxylic acids ranging from C4 to C9;
reacting at least a portion of the medium-chain fatty acids or carboxylic acids in a hydrogenation reactor to produce alcohols; and
reacting at least a portion of the alcohols in a dimerization or trimerization reactor to remove substantially all oxygen and produce olefins and to dimerize and trimerize such olefins to produce a dimerization/trimerization product comprising n-paraffins.

22. The method of claim 21 wherein the hydrogenation of the medium-chain fatty acids or carboxylic acids occurs by reacting the acids with an alcohol or an olefin to form a esters, and then hydrogenolyzing the ester to form alcohols.

23. The method of claim 21 wherein the dimerization/trimerization product comprises C8-C18 paraffins.

24. The method of claim 21 wherein said dimerization/trimerization product is further processed to produce transportation fuels or drop-in fuels.

25. The method of claim 21 wherein said dimerization/trimerization product is further processed to produce diesel or jet fuel or both.

26. The method of claim 21 further comprising processing said dimerization/trimerization product via hydroisomerization, hydrocracking, catalytic reforming, fractionation, distillation, or combinations thereof.

27. A method for producing hydrocarbon fuels from biomass, the method comprising:
converting at least a portion of the biomass into fatty acids or carboxylic acids by means of fermentation; and
reacting at least a portion of the fatty acids or carboxylic acids to remove substantially all oxygen and produce n-paraffins by means of catalytic hydrodeoxygenation or decarboxylation or both.

28. The method of claim 27 wherein said hydrodeoxygenation or decarboxylation takes place at a temperature in the range of from 0° C. to 600° C.

29. The method of claim 27 wherein said hydrodeoxygenation or decarboxylation takes place at a temperature in the range of from 200° C. to 500° C.

30. The method of claim 27 wherein said hydrodeoxygenation or decarboxylation takes place at a pressure in the range of from 0.1 to 250 atm.

31. The method of claim 27 wherein said hydrodeoxygenation or decarboxylation utilizes a catalyst comprising cobalt, molybdenum, nickel, palladium, ruthenium, or combinations thereof.

32. The method of claim 27 wherein said hydrodeoxygenation or decarboxylation product is further processed to produce transportation fuels or drop-in fuels.

33. The method of claim 27 wherein said hydrodeoxygenation or decarboxylation product is further processed to produce diesel or jet fuel or both.

34. The method of claim 27 further comprising processing said hydrodeoxygenation or decarboxylation product via hydroisomerization, hydrocracking, catalytic reforming, fractionation, distillation, or combinations thereof.
